(19) United States
(12) Patent Application Publication
(10) Pub. No.: US 2021/0164305 A1
(43) Pub. Date: Jun. 3, 2021

(54) DRILLING FLUID MEASUREMENTS USING ACTIVE GAS DILUTION

(71) Applicant: HALLIBURTON ENERGY SERVICES, INC., Houston, TX (US)

(72) Inventor: Mathew Dennis ROWE, Spring, TX (US)

(21) Appl. No.: 16/702,360

(22) Filed: Dec. 3, 2019

(51) Int. Cl. E21B 21/06; G01F 1/74; G01N 33/00; B01D 19/00

(52) U.S. Cl. CPC E21B 21/067; G01F 1/74; E21B 47/06; B01D 19/0005; B01D 19/0063; G01N 33/0073

(57) ABSTRACT

Aspects and features include a system and method for drilling fluid measurement using active gas dilution. Active and automatic correction of the flow of a mixture of a sample gas and a carrier gas supplied to detectors is used while making measurements. A computing device measures a physical state of the mixture, determines a gas flow rate for the mixture, and determines a corrected flow rate for the carrier gas based on the physical state of the mixture and the mixture gas flow rate. The computing device then adjusts the flow rate of the carrier gas into the drilling fluid to maintain the corrected flow rate. This control improves accuracy by reducing the introduction of contaminants from the measurement environment and allowing compositional measurement to be made well above the lower detection limits of the detectors being used for the measurements.

### TECHNICAL FIELD

The present disclosure relates generally to gas extraction and measurement to determine the composition of gasses produced in wellbore fluid during drilling operations. More particularly, although not necessarily exclusively, this disclosure relates to active, automated control of the dilution of gas samples extracted from wellbore fluid.

### BACKGROUND

A well can include a wellbore drilled through a subterranean formation. Systems to drill such a wellbore use drilling fluid or mud to assist in drilling boreholes into a surface of the earth. Drilling fluid may serve a variety of functions for a drilling system, including, but not limited to, cooling and cleaning a drill bit of the drilling system during operation, allowing a mud motor of the drilling system to convert fluid energy to mechanical energy to provide shaft rotation to the drill bit, and transporting the drill cuttings out of the borehole. The circulation of drilling fluid within a drilling borehole and the interaction between the downhole environment and the drilling fluid may affect or modify the properties of the drilling fluid. The properties of the drilling fluid may be analyzed subsequent to circulation in the borehole to determine the drilling environment of the drilling system.

### BRIEF DESCRIPTION OF THE DRAWINGS

FIG. 1 is a schematic diagram of an example of a drilling system including a degasser system for making wellbore drilling fluid measurements using active gas dilution according to at least some aspects of the present disclosure.

FIG. 2 is block diagram of the degasser using active gas dilution control in a drilling system according to some aspects of the disclosure.

FIG. 3 is block diagram of a computing device for making wellbore drilling fluid measurements using active gas dilution according to some aspects of the disclosure.

FIG. 4 is a cross-sectional schematic diagram depicting an example of some of the hardware in a degasser system making wellbore drilling fluid measurements using active gas dilution according to some aspects of the disclosure.

FIG. 5 is block diagram of an example of a degasser system using active gas dilution control according to some aspects of the disclosure.

FIG. 6 is block diagram of another example of a degasser system using active gas dilution control according to some aspects of the disclosure.

FIG. 7 is block diagram of an additional example of a degasser system using active gas dilution control according to some aspects of the disclosure.

FIG. 8 is a flowchart of a process for active gas dilution control according to some aspects of the disclosure.

### DETAILED DESCRIPTION

Certain aspects and features relate to a system that improves, and makes more accurate, the compositional measurements made on gasses in wellbore drilling fluid by actively and automatically correcting the flow of a mixture of a sample gas and a carrier gas supplied to detectors used to make the measurements. This control reduces the introduction of contaminants from the measurement environment and allows compositional measurement to be made well above the lower detection limit of the detectors in the analytical equipment being used for the measurements, resulting in higher measurement accuracy. It can also eliminate the need for pressure vents to maintain an appropriate fluid pressure level, since a controller adjusts flow continuously to maintain proper conditions for making measurements.

In some examples, a carrier gas source is connected to a flow control device that is coupled to the extractor of a degasser system. The sample gas evolves in the extractor and mixes with the carrier gas, with the combination gas being pushed or pulled to an enclosure. In the enclosure, the amount of combined gasses is measured. The combination gas then continues to the detectors of compositional measurement devices in order to determine composition. The measured flow value is fed to a computing device along with composition information to allow for closed-loop adjustment of the flow value. The computing device uses a stored set point referenced to pressure, temperature, or both, to achieve a flow based on a compositionally corrected flow and the specified number of detectors that consume the gas. The computing device adjusts the flow control device to maintain a constant flow rate within an accurate measurement range of the detectors.

In some examples, a system includes a gas flow arrangement including a flow controller, a measurement device, and an extractor, and a computing device in communication with the gas flow arrangement. The computing device includes a non-transitory memory device with instructions that are executable by the computing device so that the computing device mixes the carrier gas with the sample gas extracted from the drilling fluid to produce a combination gas. The computing device then measures a physical state of the combination gas, determines a gas flow rate of the combination gas, and determines a corrected flow rate for the carrier gas based on the physical state of the combination gas. The corrected flow rate is the flow rate that provides for optimized compositional measurements of the combination gas. The computing device adjusts a carrier flow rate of the carrier gas into the drilling fluid to maintain the corrected flow rate of the carrier gas. The physical state of the combination gas can include one or more of its chemical composition, its temperature, or its pressure within the enclosure.

In some examples, excess gas is purged from the extractor and the purge flow rate is based at least in part on the corrected flow rate of the carrier gas. A current purge flow rate can also be used to adjust the purge flow rate. Flow rates can be determined based on gas liquid ratio or a direct flow rate value can be used. The purge flow can be used to improve accuracy by providing more precise flow rate control.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative aspects but, like the illustrative aspects, should not be used to limit the present disclosure.

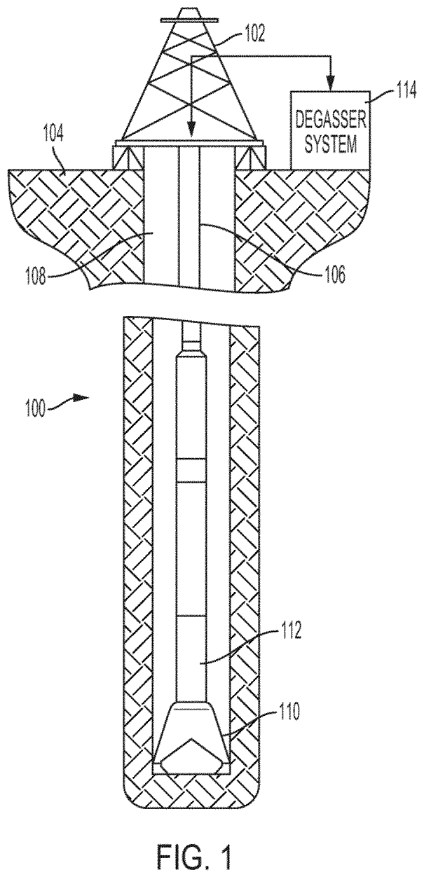

FIG. 1 is a schematic diagram of an example of a drilling system including a degasser system for making wellbore drilling fluid measurements using active gas dilution. Drilling system 100 of FIG. 1 includes a derrick 102 at a surface 104. The derrick 102 may support components of the drilling system 100, including a drill string 106. The drill string 106 may include segmented pipes that may extend below the surface 104 in wellbore 108. The drill string 106 may transmit drilling fluid (or drilling mud) necessary to operate a drill bit 110. The weight of the drill string 106 may provide an axial force on the drill bit 110. The drill string 106 includes downhole components 112 (e.g., a bottom hole assembly, a down hole motor assembly, etc.). Although FIG. 1 shows the drill bit 110 oriented in a downward direction, the drill bit 110 may be oriented in any direction in the wellbore 108.

The drilling fluid transported through the drill string 106 may be released in the wellbore 108 near the drill bit 110. The drilling fluid may serve multiple purposes, including cooling the drill bit 110 and other downhole components 112 as they rotate and interface with the surfaces of the wellbore 108 and transmitting hydraulic energy to the downhole components 112 that may be converted to mechanical energy for operation of the drill bit 110. As the drilling fluid travels through the wellbore 108 back to the surface 104, the drilling fluid may clean the wellbore 108 and may carry cuttings (e.g., rocks) excavated by the drill bit 110 to the surface 104 to be removed from the wellbore 108.

The drilling system 100 includes a degasser system 114 positioned proximate to the derrick 102 at the surface 104 of the wellbore 108. The degasser system 114 receives drilling fluid that has been circulated by the drilling system 100 in the wellbore 108. As the drilling fluid circulates in the wellbore 108 and interfaces with the downhole environment, properties of the wellbore 108 and downhole environment may be transferred to or alter the properties the drilling fluid. For example, the drilling fluid may absorb gases from formations exposed in the wellbore 108 as the drilling fluid interfaces with the surfaces of the wellbore 108 and the downhole environment. The degasser system 114 includes various devices and components for sampling and analyzing drilling fluid from the wellbore 108 to determine the properties of the wellbore 108 based on the gases absorbed during circulation of the drilling fluid in the wellbore 108. These devices include an extractor and a controller (computing device) to control the various devices in order to provide wellbore drilling fluid measurements using active gas dilution.

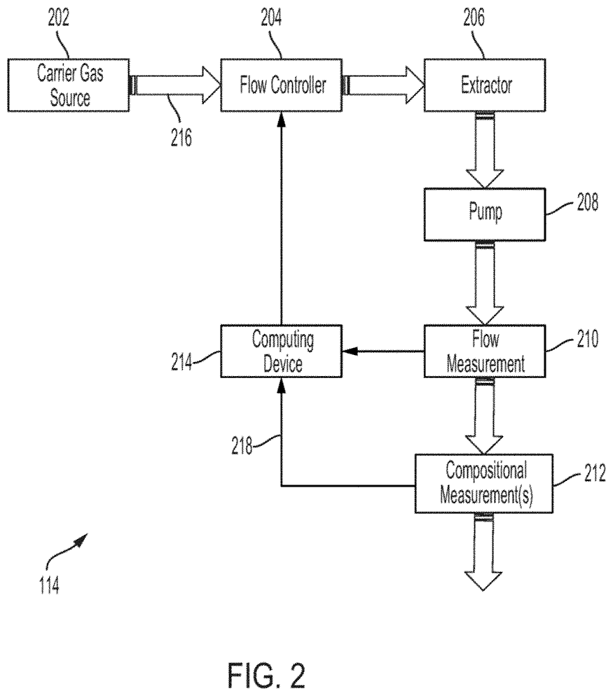

FIG. 2 is block diagram of the degasser system 114 using active gas dilution control in a drilling system like that shown in FIG. 1 according to some aspects of the disclosure. The degasser system includes a carrier gas source 202. The carrier gas may be a noble gas or a gas that is inert to the extraction and detection methodology being used for the wellbore. The carrier gas source is connected to a flow controller 204. The flow controller may be, as examples, a valve, a mass flow controller or an orifice system that can control flow. The flow controller may have flow measurement capability built in. The flow controller may also include integrated pressure and temperature measurement devices. The flow controller flows carrier gas into the extractor 206 where drilling fluid is processed to sample gas extracted from the drilling fluid. The resulting combination gas is then pumped from or pulled from the extractor by a pump 208. The pump is connected to a flow measurement device 210 that measures flow as mass or volume and may also measure temperature and pressure using integrated measurement devices. The flow measurement device 210 is connected to a compositional measurement device 212 or multiple measurement devices to determine the chemical composition of the flow. Measurement devices may include analytical instruments, detectors, transducers, or any combination of these.

Still referring to FIG. 2, the flow measurement and the current or intermediate compositional measurement(s) are sent as signals to a control system, computing device 214. In FIG. 2, arrows such as arrow 216 indicate flow of gasses or liquids, and arrows such as arrow 218 indicate electronic signals. The control system corrects the flow measurement for changes in composition, and in some examples the control system corrects for temperature and pressure. The control system will have a flow set point in some examples referencing a pressure and temperature using a stored look-up table or vector. The control system adjusts the signal to the flow controller connected to the carrier gas source to maintain the flow set point. The set point may be a specified as a gas/liquid (G/L) ratio or a flow value. If a specified G/L ratio is used, the liquid flow rate must be input or measured to provide a reference for the control system. An updated, possibly more accurate compositional measurement can then be made and the set point can be continuously updated for ongoing measurements.

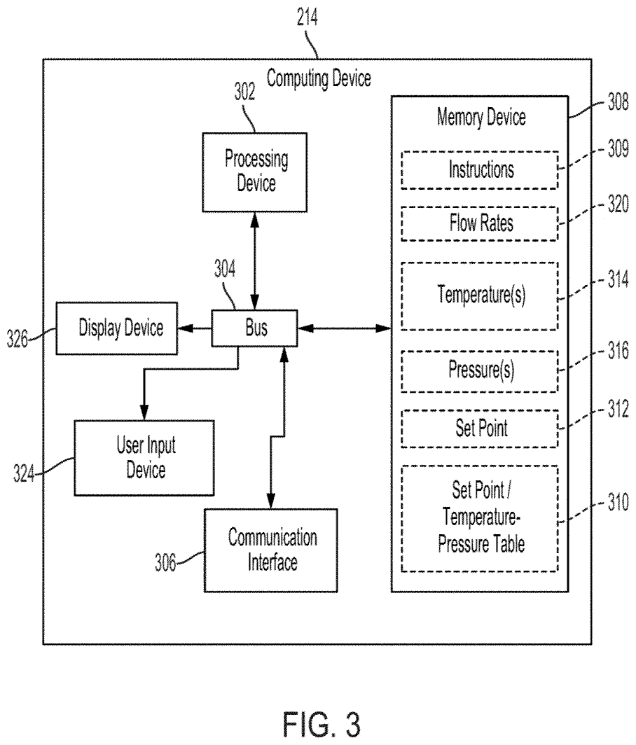

FIG. 3 is block diagram of a computing device that in some examples can serve as a control system for making wellbore drilling fluid measurements using active gas dilution according to some aspects of the disclosure. The computing device 214 includes a processing device 302, a bus 304, a communication interface 306, a memory device 308, a user input device 324, and a display device 326. In some examples, some or all of the components shown in FIG. 3 can be integrated into a single structure, such as a single housing. In other examples, some or all of the components shown in FIG. 3 can be distributed (e.g., in separate housings) and in communication with each other. The processing device 302 can execute one or more operations for active gas dilution. The processing device 302 can execute instructions stored in the memory device 308 to perform the operations. The processing device 302 can include one processing device or multiple processing devices. Non-limiting examples of the processing device 302 include a field-programmable gate array (“FPGA”), an application-specific integrated circuit (“ASIC”), a processor, a microprocessing device, etc.

The processing device 302 shown in FIG. 3 is communicatively coupled to the memory device 308 via the bus 304. The non-transitory memory device 308 may include any type of memory device that retains stored information when powered off. Non-limiting examples of the memory device 308 include electrically erasable and programmable read-only memory (“EEPROM”), flash memory, or any other type of non-volatile memory. In some examples, at least some of the memory device 308 can include a non-transitory computer-readable medium from which the processing device 302 can read instructions 309. A computer-readable medium can include electronic, optical, magnetic, or other storage devices capable of providing the processing device 302 with computer-readable instructions or other program code. Non-limiting examples of a computer-readable medium include (but are not limited to) magnetic disk(s), memory chip(s), read-only memory (ROM), random-access memory (“RAM”), an ASIC, a configured processing device, optical storage, or any other medium from which a computer processing device can read instructions. The instructions can include processing device-specific instructions generated by a compiler or an interpreter from code written in any suitable computer-programming language, including, for example, C, C++, C#, etc.

Still referring to the example of FIG. 3, the memory device 308 includes stored values for flow rates 320. The memory device 308 includes computer program code instructions 309 for controlling gas dilution. Memory device 308 in this example includes stored temperatures 314 and stored pressures 316. Memory device 308 also includes the stored, current flow set point 312 and a stored table 310 of flow set points referenced to temperatures and pressures.

In some examples, the computing device 214 includes a communication interface 306. The communication interface 306 can represent one or more components that facilitate a network connection or otherwise facilitate communication between electronic devices. Examples include, but are not limited to, wired interfaces such as Ethernet, USB, IEEE 1394, and/or wireless interfaces such as IEEE 802.11, Bluetooth, near-field communication (NFC) interfaces, RFID interfaces, or radio interfaces for accessing cellular telephone networks (e.g., transceiver/antenna for accessing a CDMA, GSM, UMTS, or other mobile communications network).

In some examples, the computing device 214 includes a user input device 324. The user input device 324 can represent one or more components used to input data. Examples of the user input device 324 can include a keyboard, mouse, touchpad, button, or touch-screen display, etc. In some examples, the computing device 214 includes a display device 326. Examples of the display device 326 can include a liquid-crystal display (LCD), a television, a computer monitor, a touch-screen display, etc. In some examples, the user input device 324 and the display device 326 can be a single device, such as a touch-screen display.

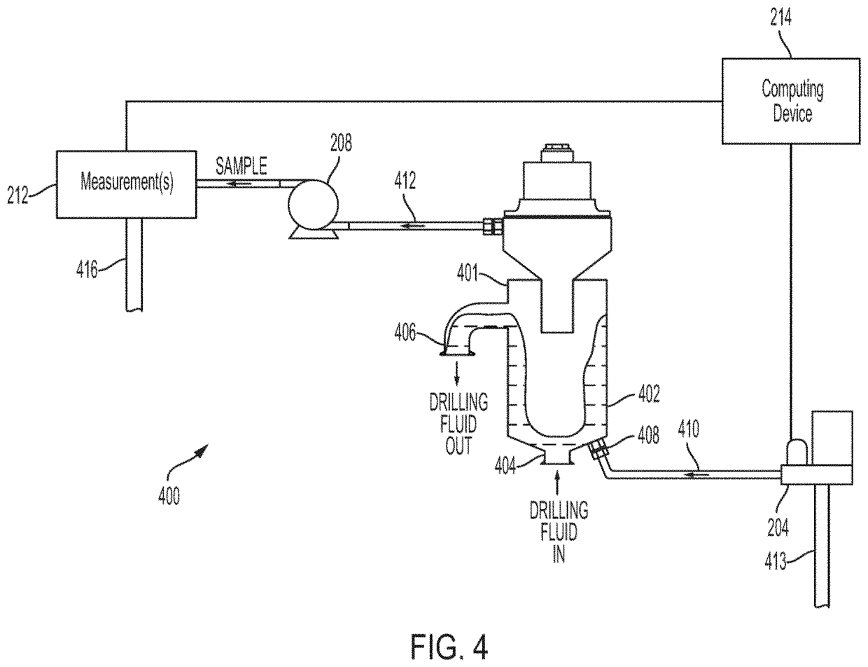

FIG. 4 is a cross-sectional schematic diagram depicting an example of some of the hardware in a degasser system making wellbore drilling fluid measurements using active gas dilution according to some aspects of the disclosure. The sample degasser system 400 includes an extractor 401 with includes a tank 402 in which drilling fluid may be located after entering through a fluid inlet valve 404 and before leaving through a fluid outlet valve 406 to allow the extractor 401 to maintain a constant volume of drilling fluid in the tank 402. The extractor 401 also includes a carrier gas intake valve 408. The carrier gas intake valve 408 may allow the carrier gas to flow directly into the tank 402. The carrier gas intake valve 408 is coupled to the flow controller 204 by a fluid line 410 that may transport the carrier gas from the flow controller 204 to the carrier gas intake valve 408. The extractor 401 is coupled to the pump 208 by the line 412. Flow controller 204 is coupled to a carrier gas source by line 413.

Still referring to FIG. 4, the pump 208 extracts the gases from the drilling fluid in the extractor 401 at an extraction rate that may be adjusted either manually or automatically, in concert with the rate of injection of the carrier gas into the extractor, to control the dilution of the gases. The gas extracted by pump 208 is a combination gas including the carrier gas and sample gas from the drilling fluid. Computing device 214 serves as a control system and is electrically connected to flow controller 204. Computing device 214 is also connected to measurement device 212, which may include detectors, analytical instruments, or both, to provide compositional measurements of the gas being pumped from the extractor 401. Gasses exit through port 416 after measurements take place.

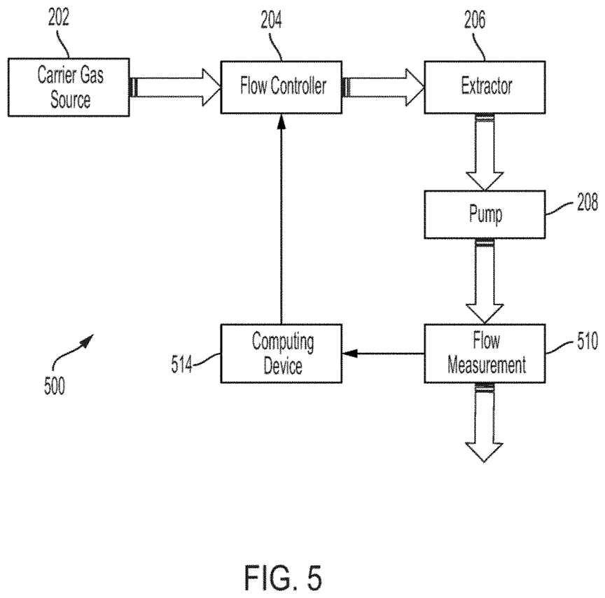

FIG. 5 is block diagram of another example of a degasser system using active gas dilution control according to some aspects of the disclosure. The main difference between degasser system 114, previously discussed, and degasser system 500 shown in FIG. 5 is that the determination of a corrected flow rate does not take into account compositional measurements. The physical state of the combination gas is based on flow, temperature, pressure, or a combination of these. The degasser system 500 includes the carrier gas source 202 and the flow controller 204. The flow controller may be, as examples, a valve, a mass flow controller or an orifice system that can control flow. The flow controller may have flow measurement capability built in. The flow controller may also include integrated pressure and temperature measurement devices. The flow controller flows carrier gas into the extractor 206 where drilling fluid is processed to sample gas from the drilling fluid. The resulting combination gas is then pumped from or pulled from the extractor by pump 208. The pump is connected to a flow measurement device 510 that measures flow as mass or volume and also measures temperature and pressure using integrated measurement devices.

Still referring to FIG. 5, the flow, temperature, and pressure measurements are sent as signals to a control system, computing device 514. The control system corrects the flow measurement for changes in temperature and pressure. The control system will have a flow set point in some examples, referencing pressure and temperature using a stored look-up table or vector. The control system adjusts the signal to the flow controller connected to the carrier gas source to maintain the flow set point.

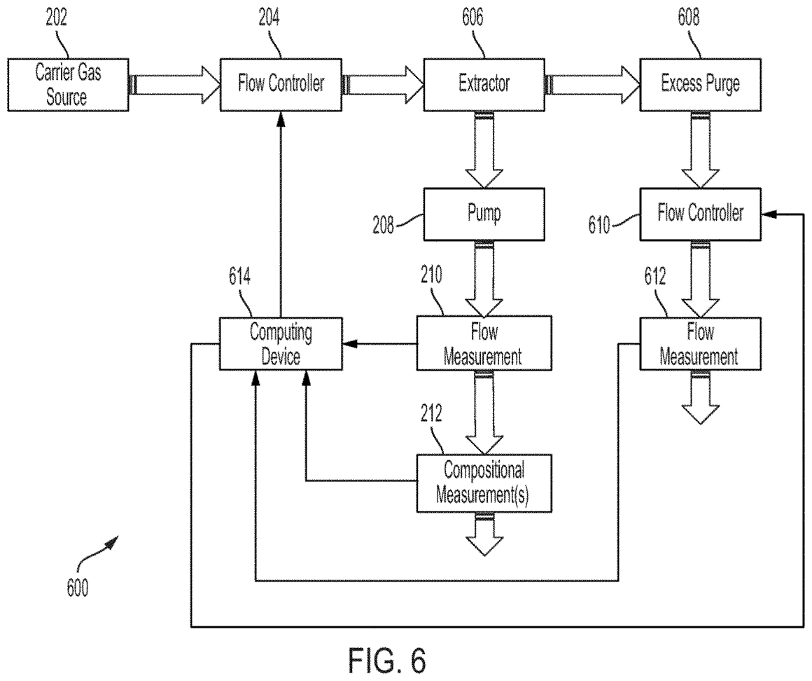

FIG. 6 is block diagram of another example of a degasser system using active gas dilution control according to some aspects of the disclosure. The degasser system 600 includes the carrier gas source 202 and the flow controller 204. The flow controller may be, as examples, a valve, a mass flow controller or an orifice system that can control flow. The flow controller may have flow measurement capability built in. The flow controller may also include integrated pressure and temperature measurement devices. The flow controller flows carrier gas into the extractor 606 where drilling fluid is processed to sample gas from the drilling fluid. Extractor 606 includes an excess purge device 608. The flow of the purge is controlled by purge flow controller 610. The combination gas from extractor 606 is pumped from or pulled from the extractor by pump 208. The pump is connected to a flow measurement device 210 that measures flow as mass or volume and may also measure temperature and pressure using integrated measurement devices.

Still referring to FIG. 6, degasser system 600 includes a flow measurement device 612 to measure the purge flow. Computing device 614 corrects the flow measurement for changes in composition, and in some examples the control system corrects for temperature and pressure. The control system will have a flow set point in some examples, referencing a pressure and temperature using a stored look-up table or vector. The control system adjusts the signal to the flow controller connected to the carrier gas source to maintain the flow set point. In system 600 however, the computing device also measures purge flow using flow measurement device 612 and controls purge flow according to a purge flow set point using purge flow controller 610. The control system can use the purge flow measurement and the corrected flow rate for the carrier gas to manage the amount of purge flow using purge flow controller 610 in order to achieve more precise control for composition measurement.

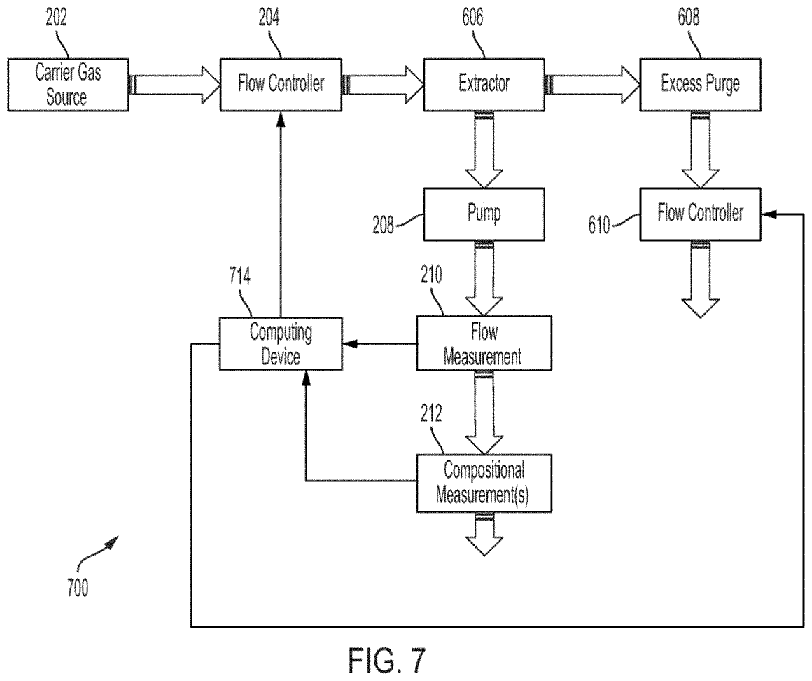

FIG. 7 is block diagram of an additional example of a degasser system using active gas dilution control according to some aspects of the disclosure. The degasser system 700 includes the carrier gas source 202 and the flow controller 204. The flow controller may be, as examples, a valve, a mass flow controller or an orifice system that can control flow. The flow controller may have flow measurement capability built in. The flow controller may also include integrated pressure and temperature measurement devices. The flow controller flows carrier gas into the extractor 606 where drilling fluid is processed to sample gas from the drilling fluid. Extractor 606 includes an excess purge device 608. The flow of the purge is controlled by purge flow controller 610. The combination gas from extractor 606 is pumped from or pulled from the extractor by pump 208. The pump is connected to a flow measurement device 210 that measures flow as mass or volume and may also measure temperature and pressure using integrated measurement devices.

Still referring to FIG. 7, computing device 714 corrects the flow measurement for changes in composition, and in some examples the control system corrects for temperature and pressure. The control system will have a flow set point in some examples, referencing a pressure and temperature using a stored look-up table or vector. The control system adjusts the signal to the flow controller connected to the carrier gas source to maintain the flow set point. In system 700 the computing device also controls purge flow according to a purge flow set point using purge flow controller 610. The control system relies on flow measurement from flow measurement device 210 and the corrected flow rate for the carrier gas to manage the amount of purge flow using purge flow controller 610 in order to achieve more precise control for composition measurement. System 700 does not have a separate purge flow measurement device.

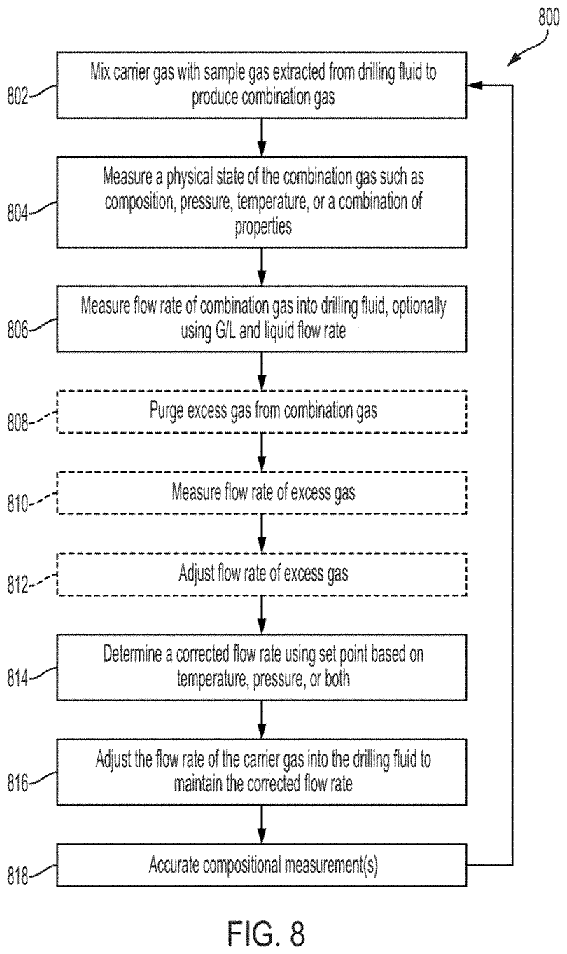

FIG. 8 is a flowchart of a process 800 for active gas dilution control according to some aspects of the disclosure. The process will be described, as an example, referencing devices in FIG. 2, FIG. 3, and FIG. 6. At block 802, carrier gas is next with sample gas extracted from the drilling fluid to produce a combination gas. At block 804, processing device 302 measures the physical state of the combination gas. This physical state can include the composition, the pressure, the temperature, or combination of such properties. For example, processing device 302 can measure the composition using compositional measurement device 212. At this stage of the processes 800, the compositional measurement performed is primarily to obtain data for adjusting the flow rate rather than to obtain an accurate picture of the drilling environment. At block 806, processing device 302 measures the flow rate of the combination gas into the drilling fluid, for example, using flow measurement device 210. The flow rate can be expressed as a numerical rate of flow or as a gas/liquid ratio.

Still referring to FIG. 8, at block 808, excess gas is optionally purged from the extractor, for example using excess purge device 608. At block 810, the flow rate of the excess gas is measured by processing device 302. At block 812, the flow rate of the excess gas is adjusted by processing device 302. At block 814, processing device 302 determines a corrected flow rate using a set point based on temperature, pressure, or both. The set point may be determined from stored table 310. At block 816, processing device 302 adjusts the flow rate of the carrier gas from carrier gas source 202 into the drilling fluid to maintain the determined, corrected flow rate. At block 818, accurate compositional measurements are made for use in analyzing the drilling fluid and the drilling environment. The process 800 can repeat continuously to maintain a set point flow rate for accurate ongoing measurements.

In some aspects, drilling fluid measurements using active gas dilution can be provided according to one or more of the following examples. As used below, any reference to a series of examples is to be understood as a reference to each of those examples disjunctively (e.g., “Examples 1-4” is to be understood as “Examples 1, 2, 3, or 4”).

Example 1. A system includes a gas flow subsystem including a measurement device and an extractor and a computing device in communication with the gas flow subsystem. The computing device includes a non-transitory memory device further including instructions that are executable by the computing device to cause the computing device to perform operations. The operations include injecting a carrier gas into the extractor to mix with a sample gas extracted from drilling fluid and produce a combination gas, acquiring, using the measuring device, a physical state of the combination gas, determining a gas flow rate of the combination gas from the extractor, and determining, based on the gas flow rate and the physical state of the combination gas, a corrected flow rate for the carrier gas that is usable for making optimized compositional measurements of the combination gas. The operations further include adjusting a carrier flow rate of the carrier gas to maintain the corrected flow rate of the carrier gas into the extractor.

Example 2. The system of example 1, wherein the physical state of the combination gas includes a chemical composition of the combination gas.

Example 3. The system of example(s) 1-2, wherein the physical state of the combination gas includes at least one of a temperature or a pressure of the combination gas.

Example 4. The system of example(s) 1-3, wherein the operation of adjusting the carrier flow rate of the carrier gas is based on a set point referenced to a temperature and a pressure.

Example 5. The system of example(s) 1-4, wherein the operations further include adjusting a purge flow controller to set a purge flow rate of excess gas from the extractor based at least in part on the corrected flow rate of the carrier gas.

Example 6. The system of example(s) 1-5, wherein the operations further include determining the purge flow rate of the excess gas.

Example 7. The system of example(s) 1-6, wherein at least one operation of determining the purge flow rate or determining the gas flow rate is based on a gas liquid ratio.

Example 8. A method includes injecting, by a processing device using a flow control device, a carrier gas into an extractor to mix with a sample gas extracted from drilling fluid and produce a combination gas and acquiring, by the processing device using a measuring device, a physical state of the combination gas. The method further includes determining, by the processing device, a gas flow rate of the combination gas from the extractor, determining, by the processing device and based on the gas flow rate and the physical state of the combination gas, a corrected flow rate for the carrier gas that is usable for making optimized compositional measurements of the combination gas, and adjusting, by the processing device, a carrier flow rate of the carrier gas to maintain the corrected flow rate of the carrier gas into the extractor.

Example 9. The method of example 8, wherein the physical state of the combination gas includes a chemical composition of the combination gas.

Example 10. The method of example(s) 8-9, wherein the physical state of the combination gas includes at least one of a temperature or a pressure of the combination gas.

Example 11. The method of example(s) 8-10, wherein adjusting the carrier flow rate of the carrier gas is based on a set point referenced to a temperature and a pressure.

Example 12. The method of example(s) 8-11 further includes adjusting a purge flow controller to set a purge flow rate of excess gas from the extractor based at least in part on the corrected flow rate of the carrier gas.

Example 13. The method of example(s) 8-12 further includes determining the purge flow rate of the excess gas.

Example 14. The method of example(s) 8-13 wherein at least one of determining the purge flow rate or determining the gas flow rate is based on a gas liquid ratio.

Example 15. A non-transitory computer-readable medium that includes instructions that are executable by a processing device for causing the processing device to perform a method. The method includes injecting a carrier gas into an extractor to mix with a sample gas extracted from drilling fluid and produce a combination gas, acquiring a physical state of the combination gas, and determining a gas flow rate of the combination gas. The method further includes determining, based on the gas flow rate and the physical state of the combination gas, a corrected flow rate for the carrier gas that is usable for making optimized compositional measurements of the combination gas, and adjusting a carrier flow rate of the carrier gas into the extractor to maintain the corrected flow rate of the carrier gas.

Example 16. The non-transitory computer-readable medium of example 15, wherein the physical state of the combination gas includes a chemical composition of the combination gas.

Example 17. The non-transitory computer-readable medium of example(s) 15-16, wherein the physical state of the combination gas includes at least one of a temperature or a pressure of the combination gas.

Example 18. The non-transitory computer-readable medium of example(s) 15-17, wherein adjusting the carrier flow rate of the carrier gas is based on a set point referenced to a temperature and a pressure.

Example 19. The non-transitory computer-readable medium of example(s) 15-18, wherein the method further includes adjusting a purge flow controller to set a purge flow rate of excess gas from the extractor based at least in part on the corrected flow rate of the carrier gas.

Example 20. The non-transitory computer-readable medium of example(s) 15-19, wherein determining at least one of the gas flow rate or the purge flow rate is based on a gas liquid ratio.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

What is claimed is:

1. A system comprising:
a gas flow subsystem including a measurement device and an extractor; and
a computing device in communication with the gas flow subsystem, the computing device including a non-transitory memory device comprising instructions that are executable by the computing device to cause the computing device to perform operations comprising:
injecting a carrier gas into the extractor to mix with a sample gas extracted from drilling fluid and produce a combination gas;
acquiring, using the measuring device, a physical state of the combination gas;
determining a gas flow rate of the combination gas from the extractor;
determining, based on the gas flow rate and the physical state of the combination gas, a corrected flow rate for the carrier gas that is usable for making optimized compositional measurements of the combination gas; and
adjusting a carrier flow rate of the carrier gas to maintain the corrected flow rate of the carrier gas into the extractor.

2. The system of claim 1, wherein the physical state of the combination gas comprises a chemical composition of the combination gas.

3. The system of claim 1, wherein the physical state of the combination gas comprises at least one of a temperature or a pressure of the combination gas.

4. The system of claim 1, wherein the operation of adjusting the carrier flow rate of the carrier gas is based on a set point referenced to a temperature and a pressure.

5. The system of claim 1, wherein the operations further comprise adjusting a purge flow controller to set a purge flow rate of excess gas from the extractor based at least in part on the corrected flow rate of the carrier gas.

6. The system of claim 5, wherein the operations further comprise determining the purge flow rate of the excess gas.

7. The system of claim 6, wherein at least one operation of determining the purge flow rate or determining the gas flow rate is based on a gas liquid ratio.

8. A method comprising:
injecting, by a processing device using a flow control device, a carrier gas into an extractor to mix with a sample gas extracted from drilling fluid and produce a combination gas;
acquiring, by the processing device using a measuring device, a physical state of the combination gas;
determining, by the processing device, a gas flow rate of the combination gas from the extractor;
determining, by the processing device and based on the gas flow rate and the physical state of the combination gas, a corrected flow rate for the carrier gas that is usable for making optimized compositional measurements of the combination gas; and
adjusting, by the processing device, a carrier flow rate of the carrier gas to maintain the corrected flow rate of the carrier gas into the extractor.

9. The method of claim 8, wherein the physical state of the combination gas comprises a chemical composition of the combination gas.

10. The method of claim 8, wherein the physical state of the combination gas comprises at least one of a temperature or a pressure of the combination gas.

11. The method of claim 8, wherein adjusting the carrier flow rate of the carrier gas is based on a set point referenced to a temperature and a pressure.

12. The method of claim 8 further comprising adjusting a purge flow controller to set a purge flow rate of excess gas from the extractor based at least in part on the corrected flow rate of the carrier gas.

13. The method of claim 12 further comprising determining the purge flow rate of the excess gas.

14. The method of claim 13 wherein at least one of determining the purge flow rate or determining the gas flow rate is based on a gas liquid ratio.

15. A non-transitory computer-readable medium that includes instructions that are executable by a processing device for causing the processing device to perform a method comprising:
injecting a carrier gas into an extractor to mix with a sample gas extracted from drilling fluid and produce a combination gas;
acquiring a physical state of the combination gas;
determining a gas flow rate of the combination gas;
determining, based on the gas flow rate and the physical state of the combination gas, a corrected flow rate for the carrier gas that is usable for making optimized compositional measurements of the combination gas; and
adjusting a carrier flow rate of the carrier gas into the extractor to maintain the corrected flow rate of the carrier gas.

16. The non-transitory computer-readable medium of claim 15, wherein the physical state of the combination gas comprises a chemical composition of the combination gas.

17. The non-transitory computer-readable medium of claim 15, wherein the physical state of the combination gas comprises at least one of a temperature or a pressure of the combination gas.

18. The non-transitory computer-readable medium of claim 15, wherein adjusting the carrier flow rate of the carrier gas is based on a set point referenced to a temperature and a pressure.

19. The non-transitory computer-readable medium of claim 15, wherein the method further comprises adjusting a purge flow controller to set a purge flow rate of excess gas from the extractor based at least in part on the corrected flow rate of the carrier gas.

20. The non-transitory computer-readable medium of claim 19, wherein determining at least one of the gas flow rate or the purge flow rate is based on a gas liquid ratio.
